(19) United States
(12) Patent Application Publication
(10) Pub. No.: US 2017/0333265 A1
(43) Pub. Date: Nov. 23, 2017

(54) ABSORBENT ARTICLE

(71) Applicant: OJI HOLDINGS CORPORATION, Tokyo (JP)

(72) Inventors: Hiroyuki Hanao, Tokyo (JP); Harue Shimizu, Tokyo (JP); Hikari Makihara, Tokyo (JP); Akira Sonoda, Tokyo (JP); Katsuhiko Sugiyama, Tokyo (JP); Ryosuke Ozaki, Tokyo (JP)

(21) Appl. No.: 15/534,211

(22) PCT Filed: Dec. 9, 2015
(86) PCT No.: PCT/JP2015/006136

§ 371 Date: Jun. 8, 2017

(30) Foreign Application Priority Data
Dec. 10, 2014 (JP) 2014-249959
Dec. 10, 2014 (JP) 2014-249960
May 15, 2015 (JP) 2015-100512

(51) Int. Cl. A61F 13/511; A61F 13/15; A61F 13/496; A61F 13/531; A61F 13/58; A61F 13/53

(52) U.S. Cl. CPC A61F 13/51108; A61F 13/496; A61F 13/51104; A61F 13/531; A61F 13/15203; A61F 2013/15422; A61F 2013/530481; A61F 2013/5315; A61F 13/58

(57) ABSTRACT

An absorbent article is provided, which effectively utilizes the entire absorber by quickly diffusing a body fluid, and reduces discomfort in a wearer by suppressing unpleasant dampness and twisting after absorption of the body fluid. An absorbent article of the present invention includes a liquid permeable top surface sheet, a liquid impermeable back surface sheet, and an absorber disposed between the top surface sheet and the back surface sheet. A top surface sheet side of the absorbent article is provided with extending a plurality of compressed lines in each of which a plurality of recess portions formed by compressing the top surface sheet and the absorber are arranged. The plurality of compressed lines form intersection regions where the compressed lines intersect with each other. The compressed recess portions are not formed in the intersection regions.

### TECHNICAL FIELD

The present invention relates to an absorbent article, and particularly relates to a disposable diaper and an absorbent pad.

### BACKGROUND ART

Various improvements have been made to an absorbent article such as a disposable diaper (hereinafter also simply referred to as the “diaper”) for better functions and wearing feeling thereof.

In the absorbent article, an absorber absorbs most of discharged body fluids. Here, the diaper covers the abdomen, buttocks, and crotch portion, and thus is affected by various body movements, especially leg movements such as walking, rolling over, and crawling of a baby. Particularly, the absorber may be twisted, folded or broken by such various body movements. This reduces fitting of the diaper to the crotch portion, and makes the fluid easily leak. Therefore, there is one including an absorber embossed on its non-skin contact surface side in order to maintain the fitting, as described in Patent Literature 1, for example. Such embossing of the absorber increases the stiffness of the absorber, making the absorber resistant to twisting and breaking even under the force applied by body movements.

Meanwhile, the absorber in the disposable diaper is positioned across the entire crotch of the wearer. During urination, the urine is diffused through a top surface sheet and the absorber from around a urine discharge area. In order to facilitate the diffusion thereof, Patent Literature 2 describes a point that a crotch portion of an absorber is provided with two lines of recess portions along a longitudinal direction of the absorber and urine is diffused by the recess portions.

There is also a diaper in which the skin contact surface side of an absorber is embossed to form compressed grooves in an oblique lattice pattern extending while being tilted with respect to a direction from the front body to the back body of the diaper, as disclosed in Patent Literature 3.

By embossing the absorber as described above, fitting of the diaper to the crotch is improved and thus leakage of a body fluid is suppressed. Particularly, the absorber embossed in a pattern having obliquely extending components, as disclosed in Patent Literature 3, achieves improved fitting to the crotch by following back and forth movements of the legs and the roundness of the buttocks.

In the meantime, in such embossing, an embossing roll having an embossing pattern protruding on its surface is rotated and moved on a long absorber sheet to form an embossed pattern. During manufacture of diapers, the long absorber sheet and the like are embossed by rotating the embossing roll thereon, followed by processes such as laminating with various sheets and cutting the resultant structure into a required size.

### CITATION LIST

### Patent Literature

PTL 1: Japanese Patent Laid-Open No. 2014-69002

PTL 2: Japanese Patent Laid-Open No. 2012-143535

PTL 3: Japanese Patent Laid-Open No. 2015-16218

### SUMMARY OF INVENTION

In general, disposable diapers are often worn by those who need assistance, such as babies and the elderly who cannot take a toileting action on their own in washrooms. Therefore, replacement of the disposable diapers is performed not only by the will of the wearer himself/herself but maybe often performed at the discretion of a caretaker. A person other than the wearer himself/herself, such as a parent of a baby or a caretaker, replaces a diaper by looking at or touching the diaper swelling and bulging by absorbing urine.

Moreover, for many diapers, a mixture of pulp and super absorbent polymers (hereinafter also referred to as the “SAPs”) is used for an absorber. Thus, the diapers are likely to swell after absorbing urine because of the SAPs.

When the skin contact surface side of the absorber is not designed to diffuse the liquid as disclosed in Patent Literature 1, the urine is not quickly diffused into the entire absorber and often stays around the urine discharge area. In this case, even a small amount of urine, for example, one time of urination causes the urine discharge area of the absorber to bulge, leading to replacement of the diaper. Therefore, the diaper is discarded without effectively utilizing the entire absorber.

Meanwhile, even though the recess portions for facilitating the diffusion of urine on the absorber are provided on the skin contact surface side of the absorber, as disclosed in Patent Literature 2, the recess portions may be collapsed under the body weight of a wearer when he/she gets up, because the recess portions are formed by compressing only the absorber. Meanwhile, even when the recess portions are not collapsed, the absorber around wall surfaces of the recess portions significantly bulge when absorbing a liquid (urine), and narrows the recess portions, which make it difficult for the urine to be quickly diffused into the adjacent absorber. As a result, the urine does not move much from the vicinity of the urine discharge area.

The inventors of the present invention have found out that there is a strong relation between liquid diffusion in the absorber and the crude density of the absorber. To be more specific, if the absorber is so compressed as to have too a high density, the number of voids for storing the liquid is small, making it difficult for the absorber to absorb the liquid. On the other hand, if the density of the absorber is too low, the absorber can absorb the liquid, but the liquid is stored in the voids and thus is hindered from actively diffusing from the voids. Therefore, the urine stays in the portion where the density of the absorber is low, adjacent to the recess portions along the longitudinal direction, and makes the crotch bulging out.

When the urine is not quickly diffused into the entire absorber and stays around the urine discharge area as described above, the absorber in the urine discharge area bulges and gets damp and sticky, causing the wearer to feel uncomfortable.

Moreover, even though the diaper still can absorb more urine, a person responsible for diaper replacement replaces the diaper since the diaper is bulging around the urine discharge area. Therefore, the diaper is discarded without effectively utilizing the entire absorber, which is uneconomical.

Meanwhile, even if the person responsible for diaper replacement does not look at or touch the portion around the urine discharge area in the diaper nor replaces the diaper, the absorber bulging by absorbing moisture is more likely to get twisted and broken than when the diaper is dry. Therefore, when urine is not diffused in one time of urination and the absorber around the urine discharge area greatly bulges, the absorber may be broken by later body movement. If the urine is further discharged to the broken portion, the urine may flow out through cracks, leading to liquid leakage.

It is a first object of the present invention to provide an absorbent article which effectively utilizes the entire absorber by quickly diffusing a body fluid, suppresses unpleasant dampness and twisting after absorption of the body fluid, and thus reduces discomfort in a wearer.

Meanwhile, in order to prevent grooves from being collapsed even under the pressure of the body weight of the wearer, the inventors of the present invention have formed compressed grooves resistant to collapse, by compressing a top surface sheet (top sheet) and an absorber together from the top surface sheet side disposed on the skin contact surface side of the absorber. In this case, again, the compressed grooves are formed by rotating an embossing roll on the absorber with the top surface sheet laminated thereon, as described above. When the compressed grooves are formed as described above, the rotating embossed roll tucks the top surface sheet and the like on top of the absorber into the grooves, and the top surface sheet and the like tend to be stretched very taut. Particularly, the emboss pattern having components extending in the oblique directions, such as the oblique lattice pattern, includes portions where the distance between simultaneously compressed points is short (interval is small), such as around lattice points. When the distance between the simultaneously compressed points is short, a strong pulling force acts on the top surface sheet from both the adjacent compressed points, leading to a very taut state of the top surface sheet.

The compressed grooves themselves are located at the position lower than uncompressed portions, and thus do not directly touch the skin of the wearer. However, the vicinities of the lattice points, i.e., the vicinities of the lattice corners are fixed in a state where the top surface sheet and the absorber are pulled and very taut. Therefore, powder super absorbent polymer (SAP) in the absorber may come into contact with the top surface sheet. In this case, the vicinities of the lattice corners in the skin contact surface of the top surface sheet may be harder than the other portions, and produce rough feeling because of a rough texture of the super absorbent polymer.

The vicinity of the lattice corner described above is located at the position higher than the bottom of the compressed groove, and thus touches the skin of the wearer and gives hard and rough feelings to the wearer, which is not preferable. Particularly, since the diaper touches a sensitive skin portion such as the crotch, a soft texture is desired. Therefore, the hard lattice corner portion gives unpleasant feelings when touching the skin.

It is a second object of the present invention to provide an absorbent article in which an emboss pattern having components extending in oblique directions is formed by compressing a top surface sheet and an absorber together, the absorbent article giving a soft texture to a wearer also in a portion where the components extending in the oblique directions intersect with each other.

An absorbent article of the present invention includes a liquid permeable top surface sheet, a liquid impermeable back surface sheet, and an absorber disposed between the top surface sheet and the back surface sheet. A plurality of compressed lines extend on the top surface sheet side, and in each of the compressed lines, a plurality of recess portions formed by compressing the top surface sheet and the absorber are arranged. The plurality of compressed lines form intersection regions where the compressed lines intersect with each other. The compressed recess portions are not formed in the intersection regions.

In the absorbent article according to the present invention, recess portions with higher pressed density of the absorber are provided in flow channels through which a body fluid such as urine flows. Thus, a difference in pressed density of the absorber inside the flow channels enables effective use of the entire absorber by quickly diffusing and absorbing the body fluid through the flow channels even when a relatively large amount of body fluid is discharged at a time.

Moreover, the absorbent article according to the present invention can provide an absorbent article that gives a soft texture to the wearer even in the intersection region where the plurality of compressed lines intersect with each other.

### BRIEF DESCRIPTION OF DRAWINGS

FIG. 1 is a three-dimensional projection view showing an external appearance of an embodiment in which an absorbent article according to the present invention is applied to an unfolding-type disposable diaper;

FIG. 2 is a three-dimensional projection view seen from the back side, showing the lower part of a body wearing the diaper shown in FIG. 1;

FIG. 3 is a partially ruptured plan view seen from the skin contact surface side of the diaper shown in FIG. 1 in an unfolded state thereof;

FIG. 4 is a cross-sectional view of the diaper taken along the line IV-IV in FIG. 3;

FIG. 5 is a three-dimensional projection view showing the diaper shown in FIG. 1 in an unfolded and exploded state;

FIG. 6 is a partial top view seen from the top sheet side, showing an absorber portion of the diaper shown in FIG. 1;

FIG. 7 is a partially enlarged top view showing a flow channel portion in the partial top view of FIG. 6;

FIG. 8 is a cross-sectional view taken along the line VIII-VIII in FIG. 7;

FIG. 9 is a cross-sectional view taken along the line IX-IX in FIG. 7;

FIG. 10 is a view showing another example of a flow channel arrangement pattern;

FIG. 11 is a view showing another example of a flow channel arrangement pattern;

FIG. 12 is a view showing another example of a flow channel arrangement pattern;

FIG. 13 is a cross-sectional view taken along the same line as the line VIII-VIII in FIG. 7, showing another example of the diaper;

FIG. 14 is a partial top view of an absorber portion seen from the top sheet side, showing another example of the diaper shown in FIG. 1;

FIG. 15 is a partial plan view schematically showing an oblique lattice emboss pattern portion in FIG. 14;

FIG. 16 is an enlarged partial plan view showing a part of FIG. 15;

FIG. 17 is a cross-sectional view taken along the line XVII-XVII in FIG. 16;

FIG. 18 is a cross-sectional view taken along the line XVIII-XVIII in FIG. 16;

FIG. 19 is a view showing a method for forming recess portions; and

FIG. 20 is a view showing another example of the method for forming recess portions.

### DESCRIPTION OF EMBODIMENTS

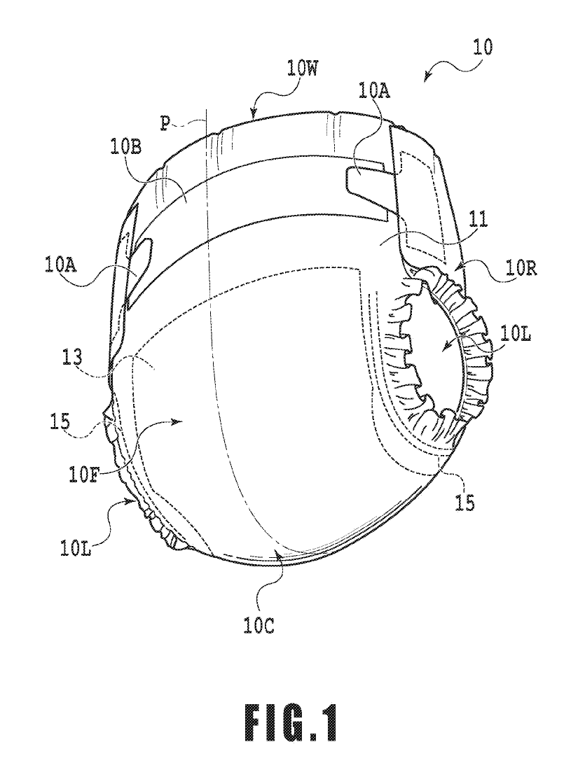

With reference to FIGS. 1 to 20, detailed description is given of embodiments of an absorbent article according to the present invention.

### First Embodiment

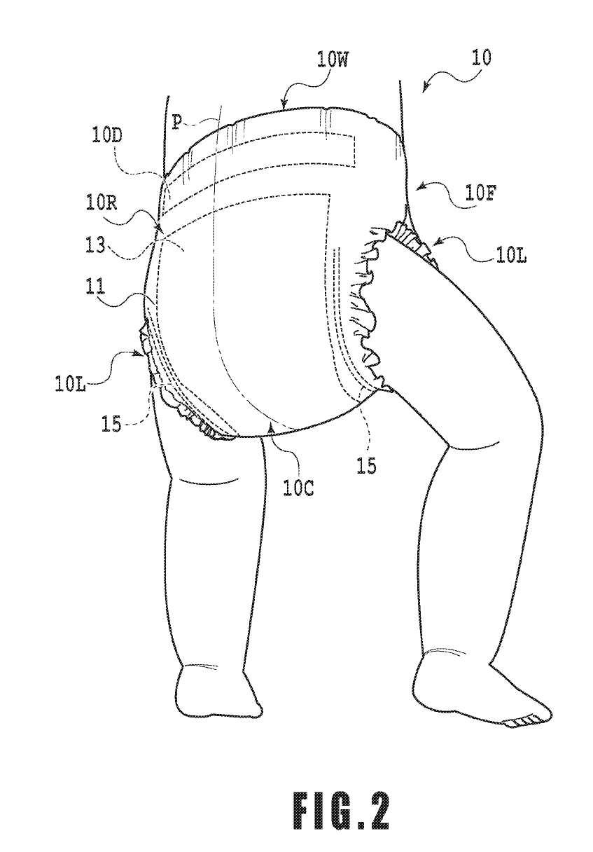

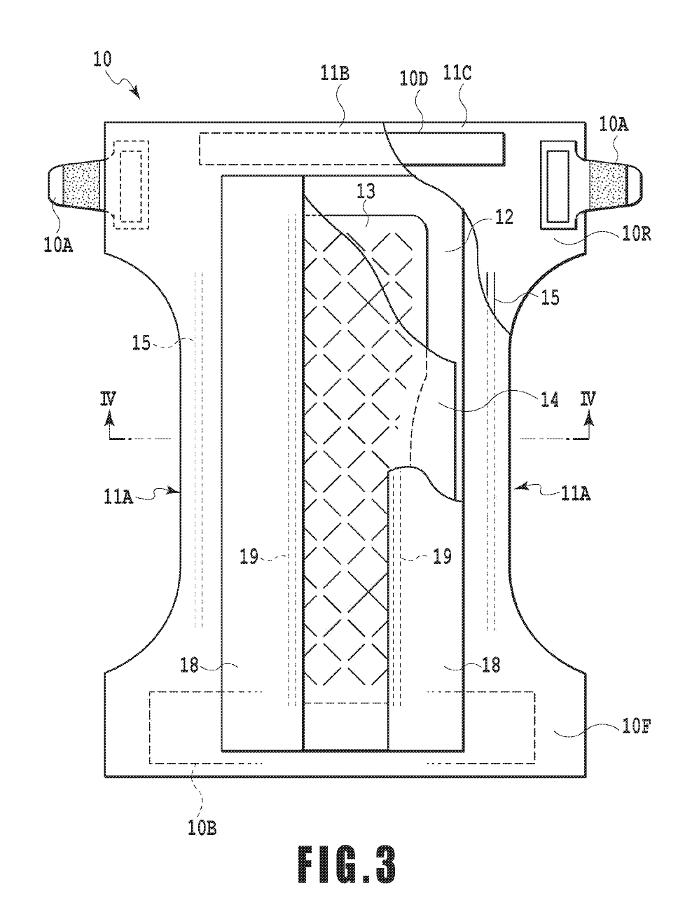

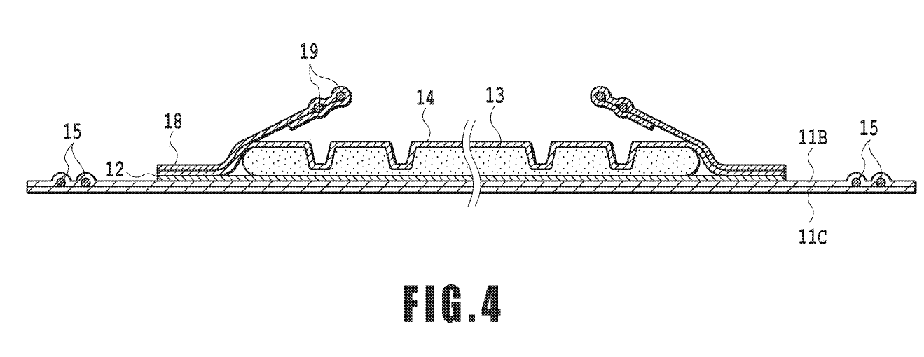

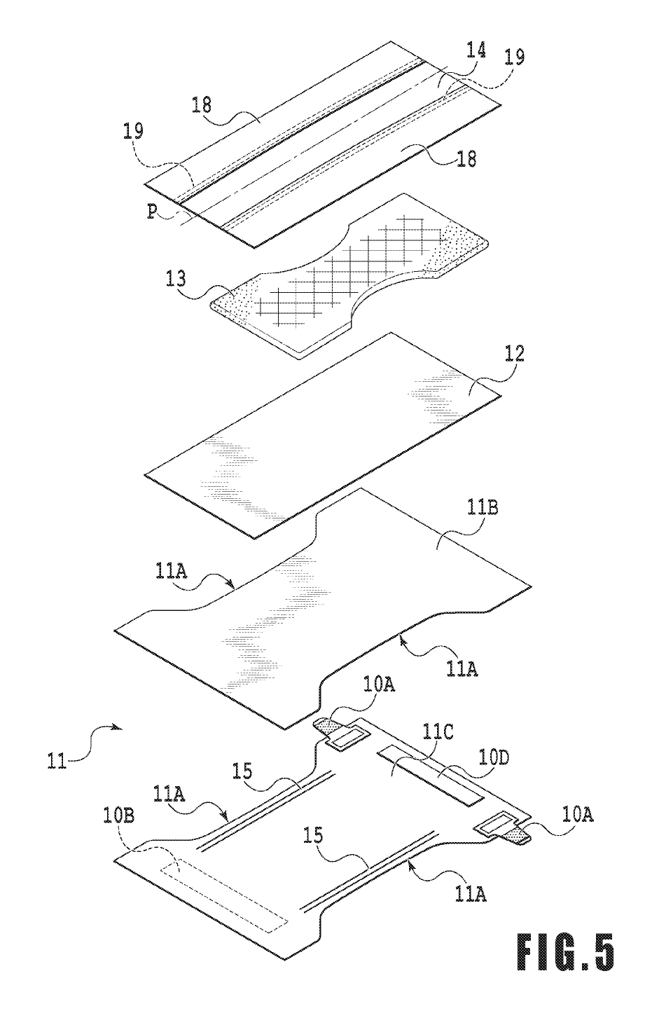

FIG. 1 is a perspective view seen from the front side and FIG. 2 is a perspective view seen from the back side, both showing an external appearance of a first embodiment in which the present invention is applied to an unfolding-type disposable diaper (so-called sheet-type disposable diaper). FIG. 2 shows a lower part of a baby's body seen from the back side when he/she is wearing the unfolding-type disposable diaper. FIG. 3 is a plan view seen from the skin contact surface side of the diaper shown in FIG. 1 in an unfolded state thereof. FIG. 4 is a cross-sectional view taken along the line IV-IV in FIG. 3. FIG. 5 is a view showing the unfolding-type disposable diaper shown in FIGS. 1 and 2 in an unfolded and exploded state.

An unfolding-type disposable diaper (hereinafter may be simply referred to as the diaper) 10 according to this embodiment includes a front body region 10F, a back body region 10R, and a crotch region 10C connecting the front body region 10F and the back body region 10R. Also, a waist opening 10W is formed by the front body region 10F and the back body region 10R surrounding a waist portion of a wearer when he/she wears the diaper. Likewise, a pair of left and right leg openings 10L are formed by lower ends of the front body region 10F and back body region 10R and the crotch region 10C surrounding thigh portions of both legs of the wearer.

As shown in FIG. 2, when the wearer wears the diaper, the front body region 10F is positioned on the abdomen side of the wearer, and the back body region 10R is positioned on the dorsal side of the wearer. The crotch region 10C covers a crotch portion of the wearer, and the legs of the wearer are inserted into the pair of left and right leg openings 10L. Therefore, the leg openings 10L are positioned at positions between the bases of the legs and the thighs of the wearer.

A line extending from the head to the crotch of the wearer along the central axis of the body when the diaper 10 is worn in a proper direction is assumed to be an imaginary line P, and is used in the following description as needed. As shown in FIGS. 1 and 2, the imaginary line P extends through the crotch portion from the abdomen side to the dorsal side in the central part of the diaper 10. To be more specific, assuming that the waist side of the diaper 10 is the top and the crotch side thereof is the bottom, for example, the imaginary line P extends in the top-bottom direction along the surface of the diaper 10 and also extends in the top-bottom direction on the dorsal side through the crotch portion. In other words, the top-bottom direction is a direction along the central axis of the body from the head to the crotch of the wearer, and the imaginary line P extends along the central axis of the body.

At both left and right ends of a cover sheet 11, which is positioned on the outer side of the diaper 10, in the back body region 10R, a pair of left and right fastening tapes 10A are attached, which connect both left and right ends of the front body region 10F by overlapping therewith when the diaper is worn, so that the leg openings 10L can be formed. The fastening tapes 10A are attached so as to be repeatedly peeled off a front patch sheet 10B attached onto the cover sheet 11 in the front body region 10F. Moreover, in an upper end portion of the cover sheet 11 in the back body region 10R, an elastic sheet 10D extending along a width direction of the cover sheet 11 is attached to impart a moderate wearing feeling to the wearer around his/her waist.

As shown in FIGS. 3 to 5, the diaper 10 according to this embodiment is formed by stacking and joining the cover sheet 11, aback sheet 12, an absorber 13, and a top sheet 14 that touches the wearer's skin in this order from outside. On both left and right sides of the cover sheet 11 in the crotch region 10C, a pair of semicircular arc-shaped cutouts 11A are formed, which serve as the leg openings 10L, respectively. The back sheet 12 which is liquid impermeable is attached to an inner cover sheet 11B, and the absorber 13 described above is disposed between the back sheet 12 and the top sheet 14 which is liquid permeable. The top sheet 14 is attached to the back sheet 12 with the absorber 13 interposed therebetween. Between an outer cover sheet 11C and the inner cover sheet 11B, which are formed of thin non-woven fabric to obtain a good texture, rubber threads 15 for forming gathering around the legs are attached in a stretched state.

On both left and right ends of the liquid permeable top sheet 14 in this embodiment, liquid impermeable side sheets 18 are attached. On inside ends of the left and right side sheets 18, rubber threads 19 for forming three-dimensional gathering are attached in a stretched state. The side sheets 18 are drawn in a longitudinal direction by stretching and contraction of the rubber threads 19 as shown in FIG. 3, thus forming the three-dimensional gathering with the inside ends lifted up as shown in FIG. 4.

Note that, in the diaper 10 according to this embodiment, the absorber 13 is elongated along the imaginary line P, and the longitudinal direction thereof is parallel to the imaginary line P. A direction perpendicular to the imaginary line P is the width direction. Note that the ratio of the length in the longitudinal direction to the length in the width direction of the diaper 10 is not limited to that of this embodiment. The ratio is changed as appropriate according to the body type of the wearer.

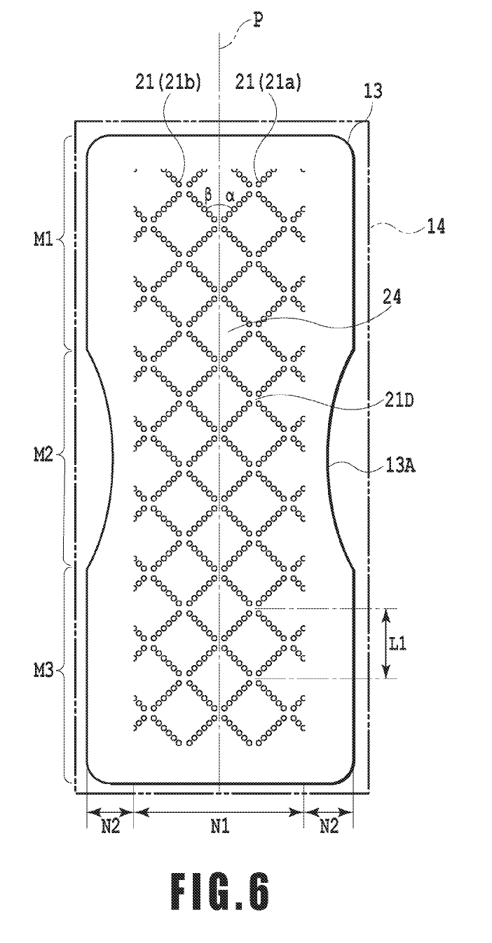

FIG. 6 is a partial top view of the crotch portion of the diaper 10 seen from the top sheet 14 side.

Note that the portion shown in FIG. 6, i.e., the top sheet 14 and the absorber 13 in the diaper 10 are also collectively called an “absorptive portion”.

The absorber 13 of this embodiment positioned below the top sheet 14 is mainly made of pulp and super absorbent polymers (hereinafter also referred to as the “SAPS”). In the absorber 13 according to this embodiment, the amount of the pulp per unit area is 185 g/m² and the amount of the SAPs per unit area is 228 g/m². The pulp and the SAPs are preferably mixed such that the ratio of the SAP weight to the absorber weight (total weight of the pulp and the SAP) is 20% to 80%. The SAPs can increase the amount of liquids absorbed without increasing the amount of the pulp. Thus, the use of the SAPs enables a large quantity of liquids to be absorbed by the thin absorber 13. However, if the amount of the SAPs is too large, gel blocks are likely to be generated by the SAPs bonding together, the SAPs swelling by absorbing the liquids, which is not preferable. On the other hand, if the amount of the SAPs is too small, the amount of liquids that can be absorbed is reduced, and thus liquid leakage is likely to occur.

The absorber 13 has an elongated shape across the front body, the crotch, and the back body, and is divided into three portions, i.e., a front body portion Ml, a crotch portion M2, and a back body portion M3. The crotch portion M2 has a pair of arc-shaped cutouts 13A formed therein corresponding to the pair of left and right leg openings 10L surrounding the thighs of the both legs. Note that the cutouts 13A do not have to be formed corresponding to the size of the absorber 13. Moreover, the absorber 13 of this embodiment has a sandglass shape with the cutouts 13A and a width narrower in the central part than at front and back ends. However, the shape of the absorber according to the present invention is not limited thereto. Assuming that the direction from the front body portion to the back body portion is the front-back (top-bottom) direction and the direction perpendicular thereto is the left-right direction, the present invention includes various shapes such as a shape having round corners at the front and back (top and bottom) ends, an oval shape extending in the front-back (top-bottom) direction, a circular shape, and a rectangular shape with approximately the same lengths in the front-back (top-bottom) and left-right directions, for example.

Meanwhile, in the diaper 10 according to this embodiment, flow channels 21 are formed by embossing in the absorptive portion that is originally a flat portion, in order to quickly diffuse a discharged body fluid such as urine from a discharge position. These flow channels 21 partition the flat portion into flat sections 24.

The flow channels 21 are formed by arranging compressed recess portions by regular embossing from the surface of the top sheet 14 toward the absorber 13, and extend in an oblique lattice pattern throughout the absorptive portion.

The flow channels 21 have the following structure.

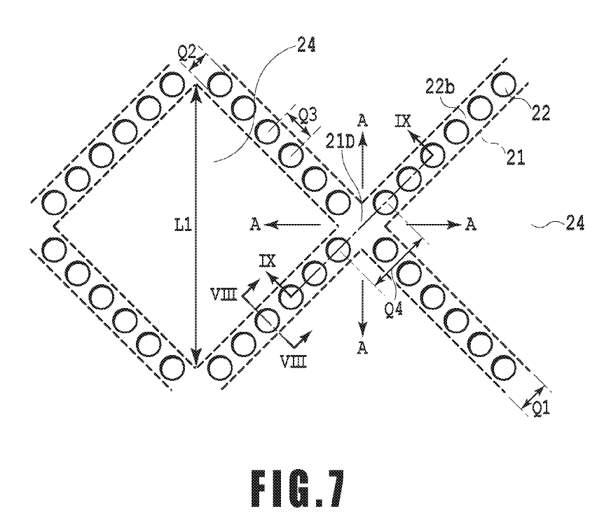

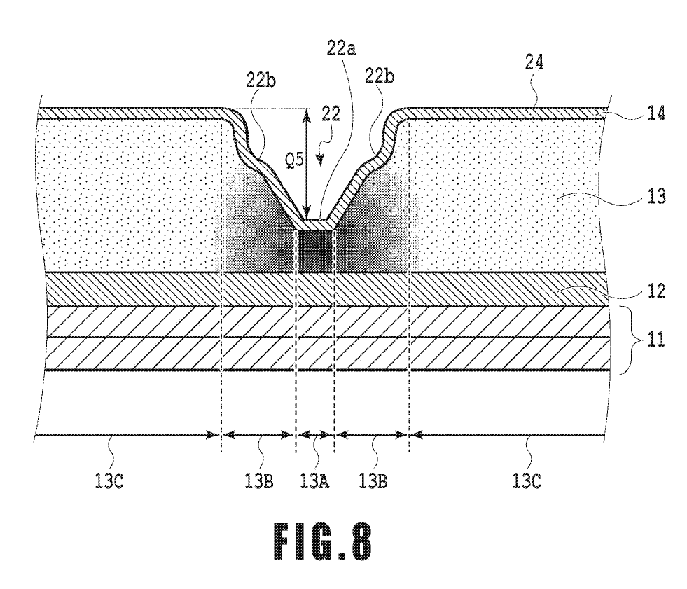

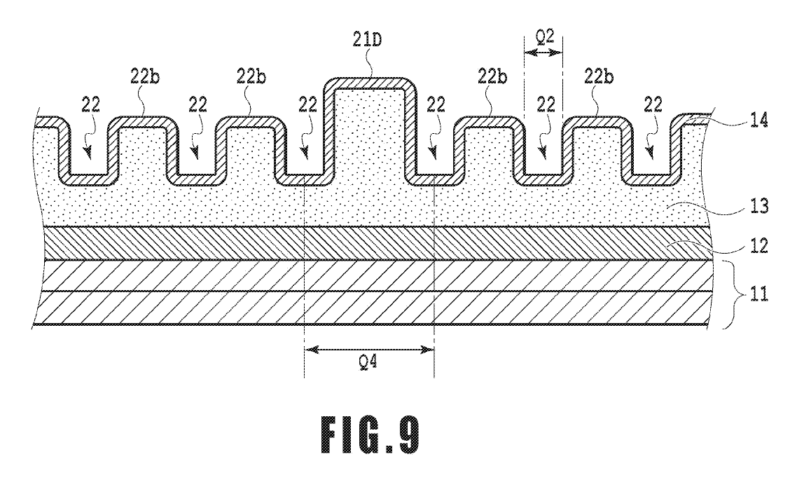

FIG. 7 is a partially enlarged view showing the flow channel 21 extending in the oblique lattice pattern. FIG. 8 is a cross-sectional view taken along the line VIII-VIII in FIG. 7. FIG. 9 is a cross-sectional view taken along the line IX-IX in FIG. 7.

As shown in FIG. 7, the flow channel 21 having a width Q1 is formed by arranging recess portions 22. The recess portions 22 are formed by intermittently compressing the top sheet 14 and the absorber 13 at the same time from the surface of the top sheet 14 in an emboss pattern such as dots. As shown in FIG. 8, when the recess portions 22 are arranged at certain intervals, the absorber 13 near the recess portions 22 is depressed, and accordingly, the absorptive portion therearound is also depressed, resulting in recess spaces 22b which are recessed from the flat section (hereinafter also referred to as the “main absorptive region”) 24 with no emboss pattern. Therefore, the flow channel 21 including the recess portions 22 and the recess spaces 22b is formed by forming the recess portions 22 at regular intervals. The recess portions 22 are formed by interposing an adhesive between the top sheet 14 and the absorber 13 and compressing the top sheet 14 and the absorber 13 at the same time from the surface of the top sheet 14. Some fibers of the top sheet 14 mesh with surface fibers of the absorber 13 to achieve firm fixation. A thickness of the absorptive portion at a bottom 22a of the recess portion 22 is about ⅙ of that when the compression is not performed. As for the compression of the absorber 13, the absorber 13 is preferably compressed to have a thickness about ⅓ to 1/7 of that when not compressed. Moreover, the thickness of the absorptive portion in the recess space 22b around the recess portion 22 is about 1/4.5 of that when the compression is not performed.

Therefore, the absorber 13 is compressed in relation to absorber density, and is divided into three portions, including a high density portion 13A with high density, i.e., the bottom 22a portion of the recess portion 22, a low density portion 13C with low density, i.e., the portion 24 without the emboss pattern, and a medium density portion 13B with medium density, i.e., a wall surface portion of the recess portion 22 and the flow channel 21. Here, it is more appropriate that the medium density portion 13B, to be more specific, is regarded as a density changing portion in which the density is not uniform and is gradually reduced from the bottom 22a to the portion 24 without the emboss pattern. In FIG. 8, the color changes according to the pressed density of the absorber 13. The pressed density gradually changes toward the main absorptive region (flat section) 24 such that the dark portion around the bottom 22a of the recess portion 22 gradually fades.

Moreover, the absorber 13 is mainly made of SAPs and pulp as described above, and pulp fiber density is mainly responsible for the density thereof. Therefore, the high density portion 13A is in a state where the pulp is compressed and there are fewer gaps between pulp fibers. On the other hand, the low density portion 13C is in a state where there are more gaps between pulp fibers than the high density portion 13A.

In addition, the recess portions 22 are formed by compressing and joining together the top sheet 14 made of synthetic fiber and the absorber 13 mainly made of pulp. Thus, the bottom 22a of each of the recess portions 22 is turned into a film, which hardly absorbs a liquid. Therefore, most of the liquid does not get absorbed and flows off on the bottom 22a of the recess portion 22. For this reason, at the bottom inside the flow channel 21, portions of the absorber 13 different in pressed density between the recess portions 22 and the recess spaces 22b depressed by the adjacent recess portions 22 alternately appear, causing the liquid to keep flowing. At the same time, the recess spaces 22b absorb the liquid.

Meanwhile, the liquid imparted onto the surface of the liquid-absorbing top sheet 14 permeates through the top sheet 14 and is absorbed by the absorber 13 in the main absorptive region (flat section) 24. On the other hand, the liquid imparted into the flow channel 21 flows off through the flow channel 21 as described above since the bottoms 22a of the recess portions 22 are in the form of film. In this flow, the liquid in the flow channel 21 is not absorbed by the bottoms 22a since the bottoms 22a of the recess portions 22 are in the form of film, and permeates into the medium density portion 13B along the top sheet 14 on the wall surfaces. Then, the liquid that cannot be secured by voids in the medium density portion 13B flows into the low density portion 13C with larger voids. In this way, the liquid moves from the medium density portion 13B to the low density portion 13C and is gradually absorbed into the main absorptive region (flat section) 24.

In the absorber 13, a portion facing a urine discharge area has the liquid directly imparted thereto also in the main absorptive region (flat section) 24, and thus sufficiently absorbs the liquid. However, a portion not facing the urine discharge area does not have the liquid directly imparted thereto, and thus absorbs the liquid flowing through the top sheet 14 or the absorber 13 from the urine discharge area. Here, the flow channel 21 plays a role of letting the liquid flow far from the portion facing the urine discharge area. More specifically, in the flow channel 21, the recess portions 22 serving as the high density portions 13A that hardly absorb the liquid are formed at regular intervals. Thus, the liquid flows through the flow channel 21 and is diffused farther from the urine discharge area into the entire absorber 13. Here, although the description is given of the case with the urine as an example, it is needless to say that the same diffusion effect can be achieved with other body fluids such as menstrual blood.

Therefore, the discharged liquid is spread within the absorptive portion by quick diffusion in the surface layer where the liquid flows through the flow channel 21 and by slow diffusion of the liquid permeating into the absorber 13 and moving slowly through the fibers of the absorber 13. As described above, the flow channel 21 is formed by compressing together the absorber 13 and the synthetic fiber top sheet 14 so that the bottom 22a is turned into a film, rather than being formed simply by compressing only the absorber 13. Thus, the flow channel 21 is made firm and suppressed from being collapsed under load. Moreover, the bottom 22a is in the form of film and hardly absorbs any liquid. Therefore, swelling by liquid absorption does not clog the flow channel 21, thus improving the diffusivity.

In order to quickly diffuse the liquid far from the crotch portion M2 facing the urine discharge area, the flow channels 21 preferably spread in every direction. In this embodiment, the flow channels 21 extend in a band pattern in the longitudinal direction of the absorber 13 from the crotch portion M2 as shown in FIG. 6. Thus, the liquid can be quickly moved to the abdomen side and the dorsal side of the absorptive portion.

Meanwhile, since a large quantity of urine is discharged at a time in urination, only the diffusion by the flow channels 21 is not sufficient, and the urine has to be absorbed in the main absorptive region (flat sections) 24. However, the absorber 13 contains the SAPs as described above. Therefore, when the absorber 13 absorbs the liquid, the SAPs swell and the swelling SAPs bond together, thus turning the absorber 13 into a gel. In a portion turned into a gel, the gel serves as a wall to inhibit the liquid permeation into the absorber 13 positioned beyond the wall. Therefore, if the flat section 24 is too small, the absorber 13 within the section is completely turned into a gel, which hinders the diffusion of the liquid into the adjacent flat sections 24 within the absorber 13, i.e., the slow diffusion by the absorber 13.

Therefore, the area of each of the flat sections 24 is preferably 0.5 cm² to 50.0 cm², more preferably 2 cm² to 18 cm². In order to realize such an area range, the length of a diagonal line L1 in the flat section 24 shown in FIG. 7 is 2<L1<6. With this size, the flat sections 24 can be utilized, and the urine does not stay only in the urine discharge area.

When the area of each flat section 24 is 2 cm² or less, the intervals between the flow channels 21 are narrow. Thus, the flow channels 21 that feel stiffer than the flat sections 24 touch the skin, making the user feel uncomfortable. Moreover, the whole absorber 13 within the flat sections 24 in the crotch portion M2 is turned into a gel, and thus no more urine can be absorbed. Furthermore, gel blocks inhibit the slow diffusion into the adjacent absorber 13.

On the other hand, when the area of each flat section 24 is too large, the diffusion by the flow channels 21 is rough, and there are more portions where each flat section 24 cannot be sufficiently utilized. Therefore, the absorber 13 cannot be efficiently used, which is not preferable.

Furthermore, as shown in FIG. 6, the flow channels 21 extend in an oblique lattice pattern throughout the absorber 13, and thus include intersection points (intersection regions in the flow channels 21) 21D. The liquid is further dispersed and spread in various directions by the flow channels 21 branched at the intersection points 21D. In order to make the diffusion by the flow channels 21 more efficient, no recess portions 22 are located at the intersection points 21D in the flow channels in this embodiment (see FIGS. 7 and 9). Since each of the intersection points 21D in the flow channels faces four main absorptive regions (flat sections) 24, a significant diffusion effect is achieved if the liquid flows into the respective main absorptive regions (flat sections) 24 from the intersection point 21D. Therefore, an environment where the liquid is more readily absorbed into the main absorptive regions (flat sections) 24 is obtained by providing no recess portions 22 at the intersection points 21D. More specifically, as shown in FIG. 9, the intersection point 21D has a lower pressed density of the absorber 13 than the bottom 22a of the recess portion 22, and thus absorbs the liquid. Furthermore, since the liquid moves to the main absorptive regions (flat sections) 24 with lower pressed density of the absorber 13, the liquid that has reached the intersection point 21D is diffused into the main absorptive regions (flat sections) 24 facing the intersection point 21D as indicated by the arrows A. Since the intersection point 21D faces four main absorptive regions (flat sections) 24, the liquid can be widely and quickly diffused and absorbed. Moreover, the adjacent main absorptive regions (flat sections) 24 are not separated from each other by the recess portion 22 in the intersection point 21D portion. Thus, liquid absorption and diffusion by the absorber 13 can be expected.

More specifically, in the portion facing multiple (four) main absorptive regions (flat sections) 24, such as the intersection point 21D, the respective absorptive regions 24 can be effectively utilized by quickly diffusing and absorbing the liquid in four directions. As shown in FIG. 6, at least one side, mostly four sides of the main absorptive region (flat section) 24 are surrounded by the flow channels 21. The liquid moves into the main absorptive regions (flat sections) 24 from the medium density portions 13B which are the wall surfaces of the flow channels 21. The additional entrance of the liquid from the intersection points 21D can increase the number of spots from which the liquid enters. When the liquid enters into the main absorptive regions (flat sections) 24 from many directions, the entire main absorptive regions (flat sections) 24 can be effectively utilized. Since the liquid can enter from many directions as described above, the liquid can be sufficiently absorbed up to the central portion of each of the main absorptive regions (flat sections) 24 even if the area of each section is increased, compared with the case where the recess portions 22 are provided at the intersection points 21D.

As described above, by extending the flow channels 21 throughout the absorber 13 in the diaper 10, the urine can be quickly diffused across the absorber 13 through the flow channels 21 from the urine discharge area, thus preventing the urine from staying only in the urine discharge area. In addition, the recess portions 22 are scattered at regular intervals, rather than being connected, in the flow channels 21. This also allows the liquid to flow while being partially absorbed at the bottom of the flow channel 21. In other words, two-layer diffusion is achieved, including liquid diffusion in the upper layer from the top sheet 14 side, i.e., the flow channel 21 side and liquid diffusion in the lower layer from the absorber 13 side. Moreover, a liquid diffusion rate in the flow channel 21 (i.e., the upper layer) is much higher than that in the absorber 13 (i.e., the lower layer). Therefore, the liquid is quickly moved by using the flow channels 21, thus preventing a large quantity of liquid from staying in a specific spot.

In addition, by providing no recess portions 22 at the intersection points 21D which are branch points, the urine is quickly absorbed into four main absorptive regions (flat sections) 24 adjacent to the intersection point 21D. Therefore, the urine discharged in the urine discharge area can be quickly moved far from the urine discharge area through the flow channels 21. At the same time, the urine is gradually absorbed into the adjacent main absorptive regions (flat sections) 24 from the wall surfaces of the flow channel 21 through which the urine is being moved. Moreover, the urine is not only branched into another flow channel 21 intersecting at each intersection point 21D that appears in the middle of the channel but also diffused into the adjacent main absorptive regions (flat sections) 24. In this way, the discharged urine is quickly diffused in various directions from the urine discharge area. Thus, the urine is absorbed in the wide region of the absorber 13 without staying in the urine discharge area. Therefore, the diaper can be prevented from being replaced, even though the whole absorber 13 still has the absorption power, when the absorber 13 around the urine discharge area is significantly swollen at one time of urination. In addition, since no urine stays in the urine discharge area, unpleasant dampness in the urine discharge area can be suppressed. Moreover, since the area of each of the main absorptive regions (flat sections) 24 can be increased, most of the surface that comes into contact with the skin can be formed as the surface of the main absorptive region (flat section) 24, which is smooth and soft without emboss pattern. Therefore, the urine can be quickly and widely diffused while maintaining good texture.

As described above, the liquid diffusivity is improved by dividing the absorptive portion into the flat sections 24 with the flow channels 21. At the same time, the absorbency that prevents liquid leakage can be maintained. Therefore, the diaper can be prevented from being replaced, even though the whole absorber 13 still has the absorption power, when the absorber 13 around the urine discharge area is significantly swollen at one time of urination.

More specifically, by extending the flow channels 21 throughout the absorber 13 in the diaper 10, the urine is quickly diffused across the absorber 13 through the flow channels 21 from the urine discharge area. Moreover, the flow channels 21 are formed by compressing the top sheet 14 and the absorber 13 together such that at least a part of the bottom thereof is formed into a film. Thus, since the bottom does not readily absorb the liquid, the absorber swelling by absorbing the liquid does not clog the flow channels unlike the one formed by compressing only the absorber.

Moreover, the two-layer diffusion structure including the slow diffusion through the absorber 13 realizes the diffusion using the entire absorber 13 in the thickness direction. In addition, the liquid diffusion rate in the flow channels 21 (i.e., the upper layer) is much higher than that in the absorber 13 (i.e., the lower layer). Therefore, the flow channels 21 are used to quickly move the liquid, thereby preventing a large amount of liquid from staying in a specific spot. Meanwhile, a large amount of urine from one time of urination needs to be quickly absorbed so as not to leak from the diaper 10. By setting each of the main absorptive regions (flat sections) 24 to have a predetermined area, a large amount of urine is quickly absorbed, and the entire absorber within the section is prevented from turning into a gel and then into gel blocks, thereby maintaining a diffusion action through the absorber into the adjacent flat sections.

In addition, by providing no recess portions 22 at the intersection points 21D which are the branch points, the urine is quickly absorbed in four main absorptive regions (flat sections) 24 adjacent to the intersection point 21D.

Note that, referring back to FIG. 6, the flow channels 21 in this embodiment include: first flow channels 21a extending while being tilted to one side (to the right in FIG. 6) in the width direction of the absorber 13; and second flow channels 21b extending while being tilted to the other side (to the left in FIG. 6). As for the position of the imaginary line P on the absorber 13, the imaginary line P extends from the upper end of the front body portion Ml to the lower end of the back body portion M3. To be more specific, as shown in FIG. 6, when the absorber 13 has the elongated shape, the imaginary line P extends in the longitudinal direction. The flow channels 21 extend while being tilted with respect to the imaginary line P. More specifically, with respect to the direction along the side about the imaginary line P, for example, the longitudinal direction, the first flow channels 21a are tilted at an angle α to one side and the second flow channels 21b are tilted at an angle β to the other side. The angle α and the angle β may be the same or different from each other. Moreover, in FIG. 6, the first flow channels 21a are tilted at the same angle and disposed at regular intervals. In other words, the first flow channels 21a are disposed parallel to each other at regular intervals. However, the present invention is not limited thereto, and also includes those with different intervals between grooves and those with different tilt angles of the grooves within a range in which the flat sections 24 can secure a predetermined area. The same goes for the second flow channels 21b.

Note that the absorber 13 is not limited to the elongated shape but may have a shape with equal length and breadth such as a square. In this case, again, first grooves 21a and second grooves 21b extend while being tilted with respect to the direction along the side.

Moreover, the flow channels 21 are not only those tilted but may be those extending in the direction along the side.

Furthermore, the flow channels 21 do not extend to the end of the diaper 10. If the flow channels 21 are provided up to the end of the diaper 10, the liquid may leak from the end, which is not preferable. Therefore, no flow channels are provided in the respective ends of the diaper 10, i.e., the leg openings 10L and waist openings 10W.

In addition, it is preferable that the flow channels 21 do not extend to the end of the absorber 13. Assuming that the direction from the front body portion M1 to the back body portion M3 is the front-back direction, no flow channels are formed at both front and back ends. Likewise, assuming that the left-right direction perpendicular to the front-back direction is the width direction, the absorber 13 has a flow channel formation region N1 in the longitudinal direction and in the center of the width direction and flow channel non-formation regions N2 on both sides thereof. In FIG. 6, the flow channel non-formation regions N2 are at the both ends of the absorber 13 in the width direction, while the flow channel formation region N1 extends in a band pattern from one end to the other end of the longitudinal direction indicated by the imaginary line P in the center. Moreover, the flow channels 21 do not reach the both ends of the longitudinal direction. Thus, liquid leakage through the flow channels 21 is surely prevented by providing no flow channels at the ends of the absorber 13.

Next, description is given of an example of a flow channel pattern by embossing.

As shown in FIG. 6, a flow channel pattern in this example is a lattice pattern in which dot-shaped recess portions 22 are lined up. The flow channels 21 are arranged such that the length of the diagonal line L1 (approximately equivalent to L1 shown in FIG. 7) in one square is 42.4 mm (4.24 cm). Moreover, as shown in FIG. 7, a diameter Q2 of the recess portion 22 is 2.0 mm, an arrangement interval Q3 between the recess portions 22 (distance between the centers of the adjacent recess portions 22) is 2.0 mm. Also, a distance Q4 between the centers of the adjacent recess portions 22 at the intersection point 21D is 6.0 mm.

The thickness of the absorber 13 and the top sheet 14 before embossing is 8.0 mm. As shown in FIG. 8, a depth Q5 of the recess portion 22 is about 5.0 mm.

Note that the thickness of the absorber 13 and the top sheet 14 before embossing is preferably 5.0 mm to 20.0 mm. Moreover, as for the configuration of the absorber 13 and the top sheet 14, the diameter Q2 of the recess portion 22 is preferably 1.0 mm to 4.0 mm, the arrangement interval Q3 is preferably 1.0 mm to 9.0 mm, and the depth Q5 is preferably 2.0 mm to 10.0 mm. Furthermore, the distance Q4 between the centers of the adjacent recess portions 22 at the intersection point 21D is preferably 2.0 mm to 12.0 mm.

Next, description is given of an experiment of comparison between a diaper with the flow channel pattern of this embodiment and a diaper without such a flow channel pattern.

The diaper with the flow channel pattern of this embodiment (Example 1) and the diaper without the flow channel pattern (Comparative Example 1) are attached to a doll, respectively, and are compared for the diffusivity after water is poured three times, 50 cc at a time, from the crotch portion. To be more specific, the diaper is equally divided into five regions, i.e., abdominal end, abdominal center, crotch, dorsal center, and dorsal end in the imaginary line P direction, and the weight of each region is measured after each pouring to determine the liquid diffusivity. Then, a ratio of the weight of each region to the total weight of the diaper after three times of pouring is calculated.

The total weight of the diaper before pouring is 31.7 g.

Table 1 shows the result of Example 1, while Table 2 shows the result of Comparative Example 1.

TABLE 1

| | | | | Ratio of Weight |
|---|---|---|---|---|
| | First | Second | Third | After Three |
| Example 1 | Round (g) | Round (g) | Round (g) | Times (%) |
| Abdominal | 0.3 | 3.3 | 8.0 | 5.3 |
| End | | | | |
| Abdominal | 12.2 | 30.4 | 40.3 | 26.9 |
| Center | | | | |
| Crotch | 25.3 | 33.7 | 53.0 | 35.3 |
| Dorsal | 11.9 | 29.2 | 39.7 | 26.5 |
| Center | | | | |
| Dorsal End | 0.3 | 3.4 | 9.1 | 6.0 |

TABLE 2

| | | | | Ratio of Weight |
|---|---|---|---|---|
| Comparative | First | Second | Third | After Three |
| Example 1 | Round (g) | Round (g) | Round (g) | Times (%) |
| Abdominal | 0.0 | 0.4 | 0.9 | 0.6 |
| End | | | | |
| Abdominal | 9.1 | 32.1 | 45.6 | 30.4 |
| Center | | | | |
| Crotch | 33.7 | 40.6 | 64.5 | 43.0 |
| Dorsal | 7.2 | 26.5 | 37.9 | 25.3 |
| Center | | | | |
| Dorsal End | 0.0 | 0.4 | 1.2 | 0.8 |

As can be seen from Table 1, in the diaper with the flow channel pattern of this embodiment in Example 1, the liquid is diffused to the abdominal end and the dorsal end of the absorber, and the ratio of the weight of the crotch portion is lower than that in the result of Comparative Example 1. Therefore, it can be understood that the poured water does not stay in the crotch where the water is poured, and is diffused across the absorber.

### MODIFIED EXAMPLE

In the first embodiment, the flow channels 21 are formed in the emboss pattern in which the recess portions 22 are intermittently arranged. However, the present invention is not limited thereto. The flow channels 21 may have the recess portions 22 connected therein, i.e., may be formed by continuous embossing.

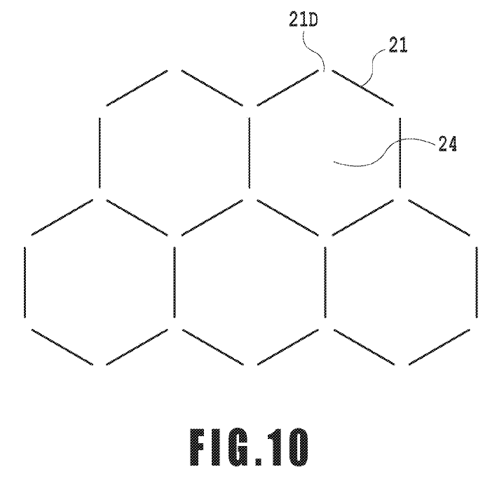

The flow channels 21 may be formed not only in the oblique lattice pattern but also in a honeycomb pattern (Modified Example 1) as shown in FIG. 10. In this case, the area of the flat section 24 surrounded by the flow channels 21 is 0.5 cm² to 50.0 cm².

In the first embodiment and Modified Example 1, no recess portions 22 are provided at the intersection points 21D in the flow channels 21. However, the present invention is not limited thereto, but the recess portions 22 may be provided.

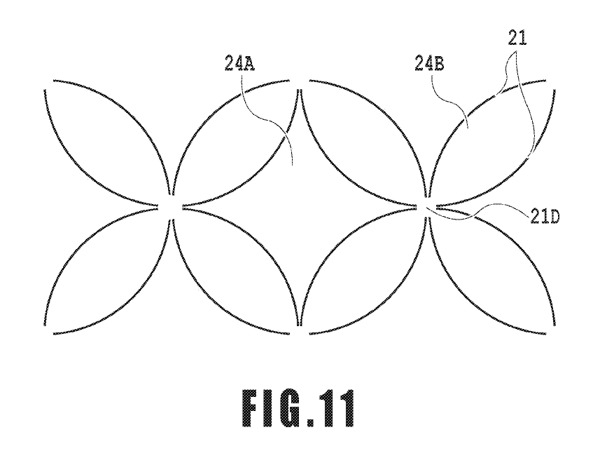

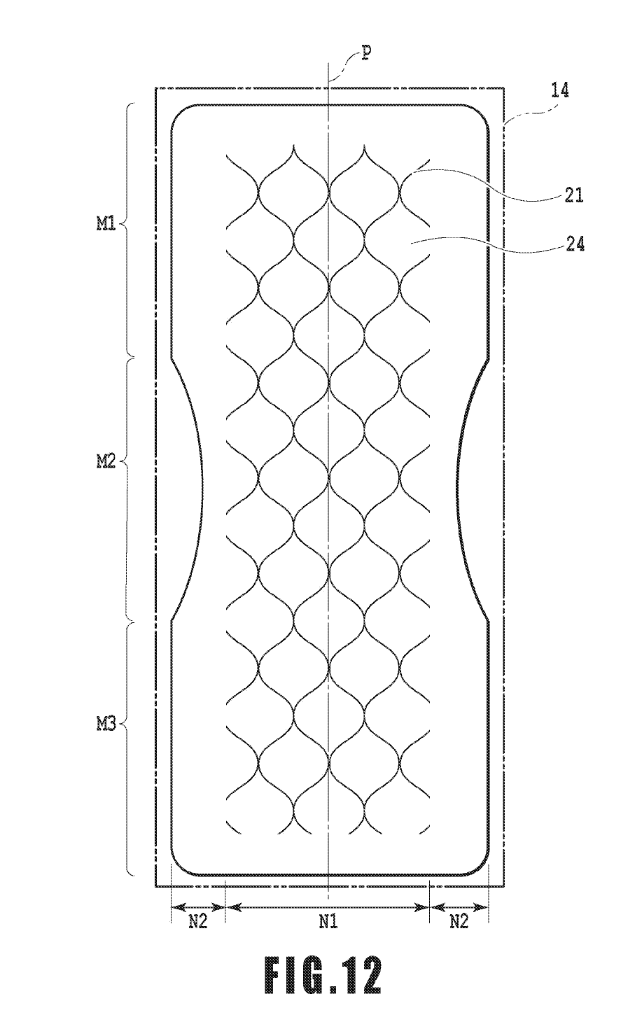

Moreover, as shown in FIGS. 11 and 12, the flow channels 21 are not limited to the linear pattern, but may be a curved pattern (Modified Examples 2 and 3). As shown in FIG. 11, a pattern may be adopted in which main absorptive regions (flat sections) 24A and 24B surrounded by curved flow channels 21 have different areas. In this case, again, no recess portions 22 are located at intersection points 21D. Thus, by forming the flow channels 21 not only in the oblique lattice pattern but also in the honeycomb pattern or curved pattern, a design effect is also achieved by shaping of the flow channels 21, thus improving aesthetic quality.

Alternatively, the flow channels 21 may be formed using an embossing mold in which lines corresponding to the flow channels 21 and dots corresponding to the recess portions 22 are combined, such that lines to be the flow channels 21 are formed by gentle compression and then portions to be the recess portions 22 are stacked thereon before compression. In this case, the portions 22b other than the recess portions 22 in the flow channels 21 are formed by compression. However, it is acknowledged that there is a difference in density between the bottom 22a and the absorber 13, due to a difference in strength of compression. Although the absorptive effect of the portions 22b other than the recess portions 22 in the flow channels 21 is reduced, the flow through the flow channels 21 can be surely secured.

Furthermore, as for the recess portions 22, not only the circular dots but also various shapes can be used, such as a semicircular shape, a rectangular shape, and a triangular shape.

In addition, the diaper is not only the one in which the top sheet 14 is disposed directly on the absorber 13, but may also be one in which the top sheet 14 is disposed on the absorber 13 after the absorber 13 is wrapped in a hydrophilic sheet 13S.

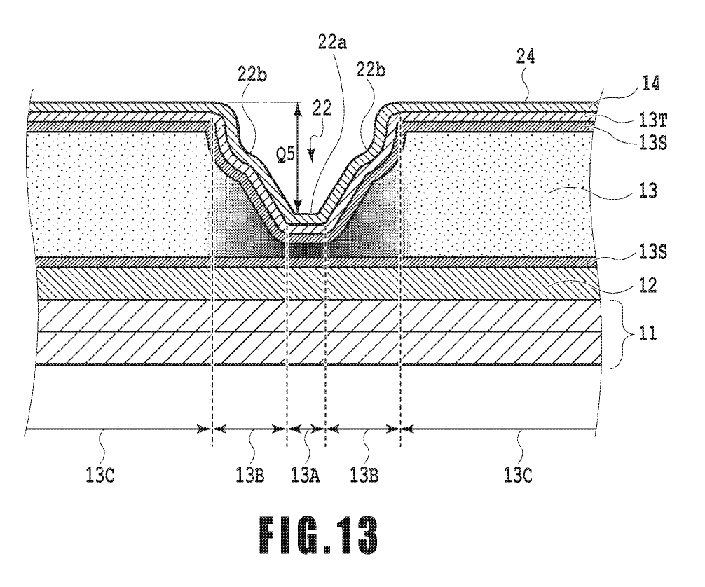

Moreover, as shown in FIG. 13, a liquid diffusion sheet 131 to improve liquid diffusivity may be provided between the top sheet 14 and the hydrophilic sheet 13S. The body fluid is more readily diffused by the liquid diffusion sheet 13T. Also, the hydrophilic sheet 13S may be attached so as to cover the absorber 13 or may be simply disposed on the top and back without covering the ends of the absorber 13. Moreover, the position of the liquid diffusion sheet 13T may be between the hydrophilic sheet 13S and the absorber 13. The body fluid is more readily diffused by the liquid diffusion sheet 13T.

### Second Embodiment

Next, description is given of a structure of an absorber portion according to a second embodiment of the present invention.

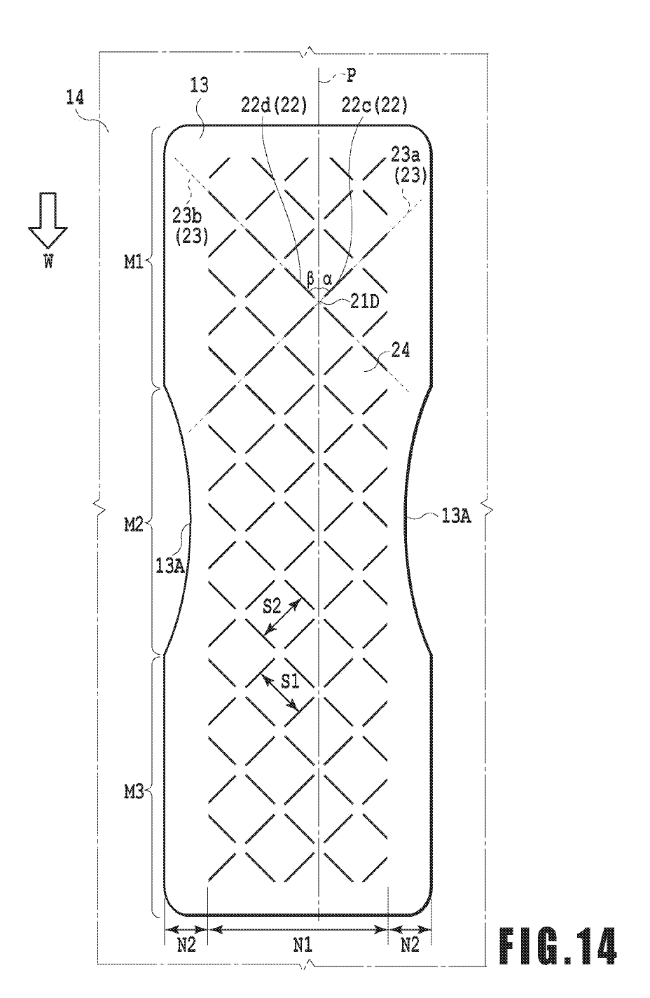

FIG. 14 is a partial top view seen from the top sheet 14 side, showing a portion where an absorber 13 and the top sheet 14 are positioned.

The absorber 13 of this embodiment positioned below the top sheet 14 is mainly made of pulp and super absorbent polymers (hereinafter also referred to as the “SAPS”). The absorber 13 has an elongated shape across the front body, the crotch, and the back body, and is divided into three portions, i.e., a front body portion Ml, a crotch portion M2, and a back body portion M3. The crotch portion M2 has a pair of arc-shaped cutouts 13A formed therein corresponding to a pair of left and right leg openings 10L surrounding the thighs of the both legs. Note that the cutouts 13A do not have to be formed corresponding to the size of the absorber 13. Moreover, the absorber 13 of this embodiment has a sandglass shape with the cutouts 13A and a width narrower in the central part than at front and back ends. However, the shape of the absorber according to the present invention is not limited thereto. Assuming that the direction from the front body portion to the back body portion is the front-back (top-bottom) direction and the direction perpendicular thereto is the left-right direction, the present invention includes various shapes such as a shape having round corners at the front and back (top and bottom) ends, an oval shape extending in the front-back (top-bottom) direction, a circular shape, and a rectangular shape with approximately the same lengths in the front-back (top-bottom) and left-right directions, for example.

Moreover, the absorber 13 is wrapped in an unillustrated core wrap (tissue). The core wrap is a hydrophilic thin sheet. Note that, although the absorber 13 wrapped in the core wrap is used in this embodiment, the absorber in the present invention does not have to be wrapped in the core wrap.

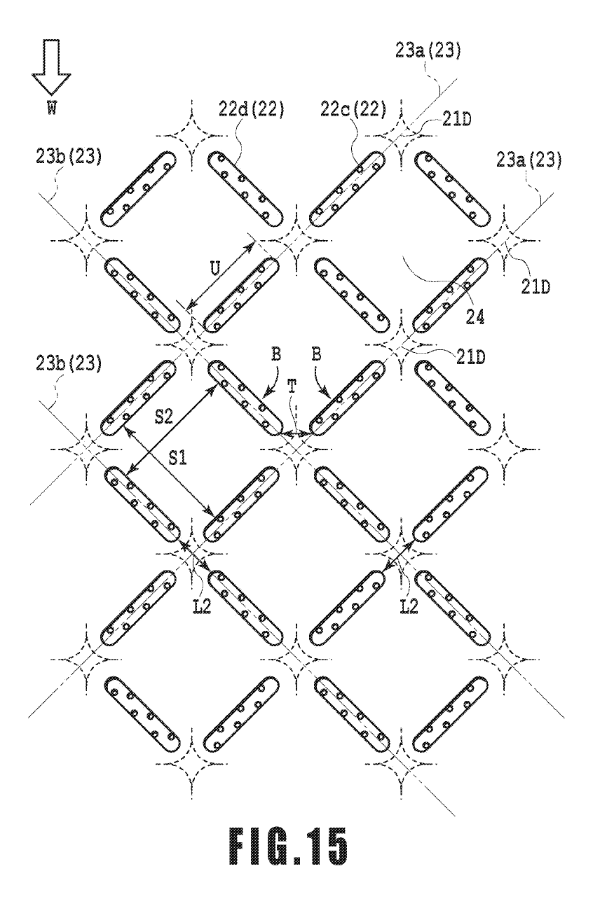

As shown in FIGS. 14 and 15, the diaper 10 has a compressed section formation region (emboss pattern formation region) N1, which is compressed by regular embossing from the surface of the top sheet 14 to the absorber 13. In the compressed section formation region N1, recess portions 22 are formed by compression, and compressed lines 23 are formed in an oblique lattice pattern when seen from a distance by the arrangement of the recess portions. The recess portions 22 in this embodiment are tilted with respect to an imaginary line P along the central axis of the body of a wearer when he/she wears the diaper 10. To be more specific, the recess portions 22 include: rightward recess portions 22c tilted to the right in FIGS. 14 and 15, which is a first direction, at a predetermined angle α with respect to the imaginary line P; and leftward recess portions 22d tilted to the left in FIGS. 14 and 15, which is a second direction, at a predetermined angle β with respect to the imaginary line P. The recess portions 22 tilted in the same direction are arranged so as to form a line at predetermined intervals L2 and thus form an emboss pattern to be an obliquely extending lattice pattern when seen from a distance. Although not shown in FIGS. 14 and 15, the recess portions 22 are formed by compressing the core wrap and the absorber 13 together from the surface of the top sheet 14.

Note that, as shown in FIG. 14, the emboss pattern is not formed at both ends of the absorber 13. Therefore, the absorber 13 has compressed section non-formation regions (emboss pattern non-formation regions) N2 positioned on both sides of the compressed section formation region (emboss pattern formation region) N1. This is to prevent a body fluid from leaking from the leg openings 10L through the emboss pattern.

As shown in FIG. 14, lines in which the rightward recess portions 22c are arranged are first compressed lines 23a, and lines in which the leftward recess portions 22d are arranged are second compressed lines 23b. These compressed lines 23 are linear lines tilted at the same angles as the tilt angles of the recess portions 22 with respect to the imaginary line P. The first compressed lines 23a are arranged in parallel with an interval S1 from each other. The second compressed lines 23b are arranged in parallel with an interval S2 from each other. The emboss pattern that is the oblique lattice pattern is formed by arranging the first compressed lines 23a and the second compressed lines 23b as described above. Although the intervals S1 and S2 are the same value in this embodiment, the both may be different values.

As can be understood from FIG. 14, the recess portions 22 each have a length to form squares in the lattice pattern. Here, the length of the recess portion 22 is shorter than the intervals S1 and S2 described above. More specifically, as described above, the recess portions 22 are arranged at predetermined intervals L2 to form the compressed lines 23. The length of the recess portion and the interval in the lattice pattern are determined such that no recess portions are positioned at intersection regions 21D in the lattice pattern. Therefore, the rightward recess portions 22c and the leftward recess portions 22d do not overlap with each other, and the top sheet 14 and the absorber 13 at the intersection regions 21D in the oblique lattice pattern formed by the recess portions 22 serve as uncompressed regions. Likewise, the top sheet 14 and the absorber 13 also serve as uncompressed regions in main absorptive regions 24 which are internal regions of the squares defined by the respective recess portions 22.

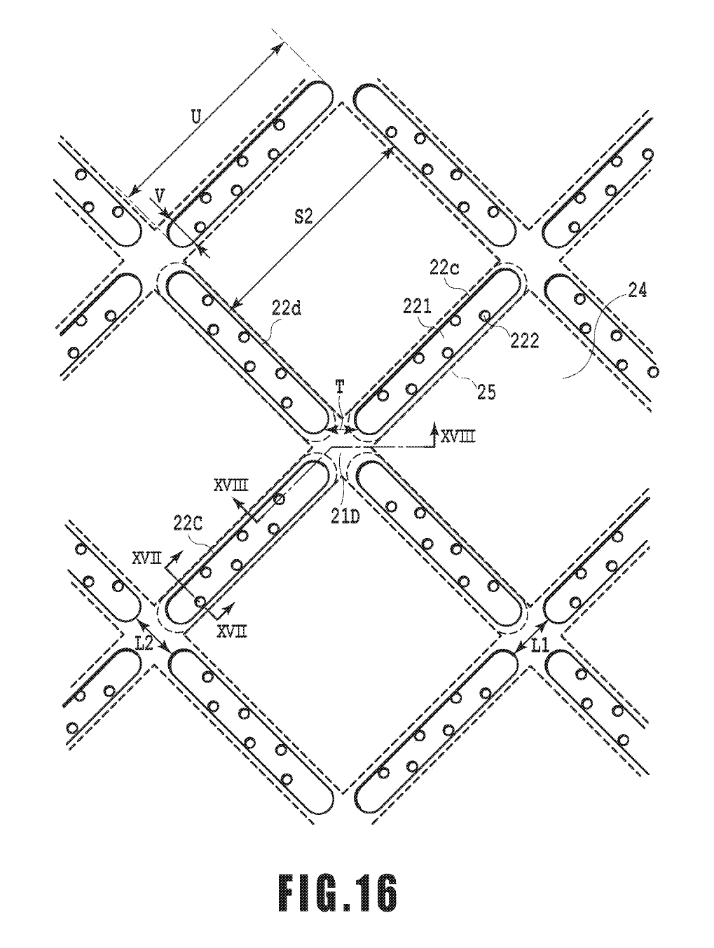

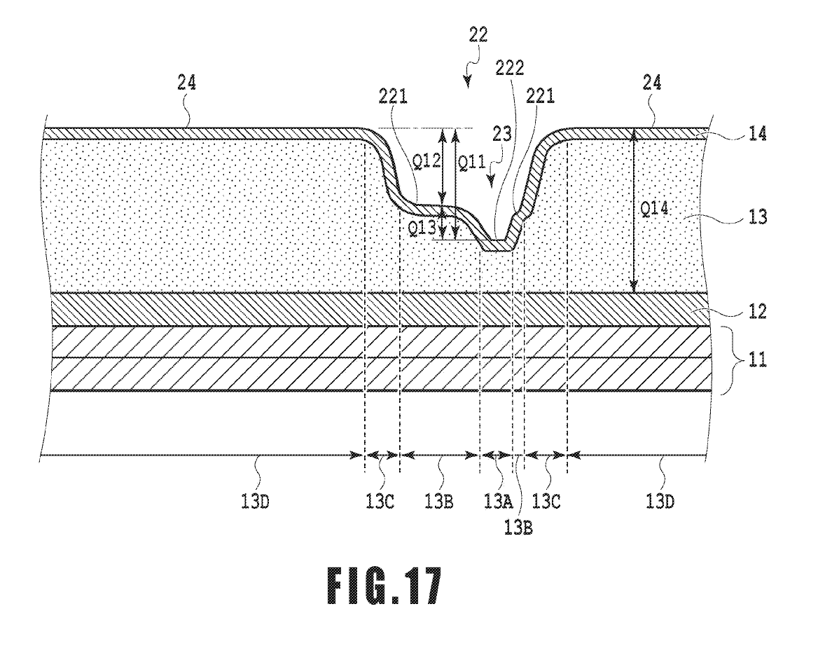

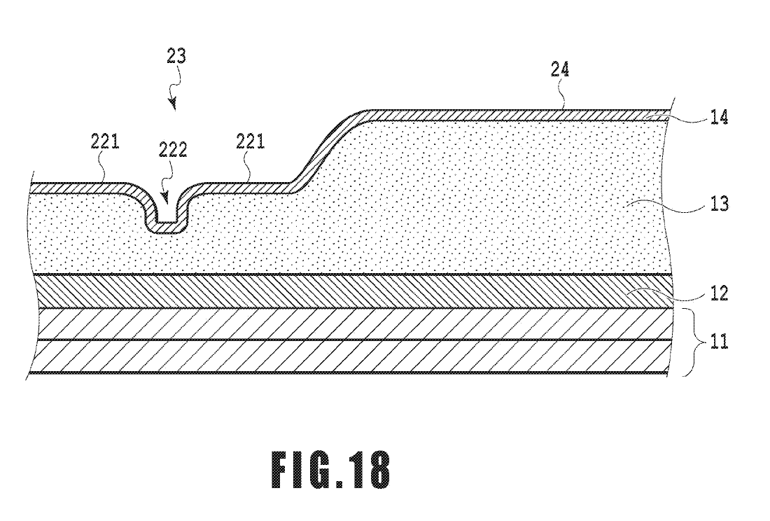

FIG. 16 is an enlarged schematic view showing the vicinity of intersection points of the compressed lines in FIG. 15. FIG. 17 is a cross-sectional view taken along the line XVII-XVII in FIG. 16. FIG. 18 is a cross-sectional view taken along the line XVIII-XVIII in FIG. 16.

As shown in FIGS. 16 and 17, the recess portion 22 includes a first recess portion 221 that forms the external shape thereof and a circular second recess portion 222 that is intermittently arranged in the first recess portion 221 and is more deeply depressed than the first recess portion 221. The first recess portion 221 has an elongated shape with a width V and a length U that is shorter than the lengths S1 and S2 of the sides of the squares in the lattice pattern, as shown in FIG. 16. To be more specific, the first recess portion 221 has arc-shaped ends. Also, inside the first recess portion 221, the circular second recess portions 222 are alternately arranged at predetermined intervals with respect to the central axis in the longitudinal direction thereof. The second recess portions 222 are depressed more deeply in the thickness direction of the absorber 13 than the first recess portion 221.

As shown in FIG. 17, in the recess portion 22, the second recess portion 222 is positioned at the deepest point and the first recess portion 221 is located at a position slightly above the bottom of the second recess portion 222. Assuming that a depth to the deepest position of the recess portion 22, i.e., the bottom of the second recess portion 222 is Q11 and a depth to the bottom of the first recess portion 221 is Q12, Q13 that is a difference obtained by subtracting Q12 from Q11 is about 3.5 to 15.0% of the depth Q11 to the bottom of the second recess portion 222. Meanwhile, assuming that a thickness of the absorber 13 and the top sheet 14 in the uncompressed region is Q14, the depth Q11 to the bottom of the second recess portion 222 is about 42.5% to 97.5% of the thickness Q14, and the depth Q12 to the bottom of the first recess portion 221 is about 37.5% to 95.0% of the thickness Q14. Thus, the recess portions 22 in this embodiment are formed into a step shape with two depths by significantly deeply compressing the absorber 13.

The embossing to form the recess portions 22 is compressing the top sheet 14 and the absorber 13 together from the surface of the top sheet 14 by interposing an adhesive between the top sheet 14 and the absorber 13. The recess portions 22 are formed by compressing the top sheet 14 and the absorber 13 together from the surface of the top sheet 14 with a predetermined mold formed in an embossing roll. The second recess portions 222 are small circles and each have a small area. Therefore, pressing with the embossing roll focuses pressure on the second recess portions 222, and thus the absorber 13 and the top sheet 14 are firmly compressed. During this compression, pulp fibers in the absorber 13 and fibers in the top sheet 14 firmly mesh with each other, and thus the both are joined in an integral state. Although the same pressing force is also applied to the first recess portion 221 during the compression, the pressure is not focused as much as in the second recess portion 222 because of a large area thereof. Therefore, the junction between the absorber 13 and the top sheet 14 is slightly weaker than that in the second recess portion 222, but is sufficient to form the recess shape. In this way, the absorber 13 is firmly compressed in the second recess portion 222, and the absorber 13 and the top sheet 14 are firmly joined, thereby maintaining the shape of the recess portion 22. For example, even when the pressure of the weight of the wearer is applied onto the surface of the absorber 13 by the wearer sitting or the like, the recess portion 22 maintains its shape without collapsing. Moreover, even when the diaper 10 is pulled hard by various movements of the legs, the top sheet 14 and the absorber 13 are firmly joined, and thus the recess portion 22 maintains its shape. Here, it is also conceivable to form the recess portion 22 by applying a strong compression force to the entire recess portion 22, rather than the two-step structure including the first recess portion 221 and the second recess portion 222. More specifically, in embossing, it is also conceivable to perform compression using a mold with only approximately oval protrusions, each having a flat surface, rather than using a mold in which a round protrusion corresponding to the second recess portion 222 further protrudes inside an approximately oval protrusion corresponding to the first recess portion 221. However, such a mold has no spot where the pressure is focused. Therefore, a spot where the top sheet 14 and the absorber 13 are joined in a state where the fibers thereof mesh with each other cannot be created unless strong pressure is applied to the whole. For this reason, a very strong pressing force is required, and the top sheet 14 may be broken. Moreover, by compression with weak pressing force to the whole, the recess portions 22 that can withstand the weight and various movements of the wearer cannot be formed. In this embodiment, by providing protrusions corresponding to the second recess portions 222 in the embossing mold and partially firmly compressing the top sheet 14 and the absorber 13, spots where the top sheet 14 and the absorber 13 are firmly joined are created, and occurrence of defects such as the top sheet 14 being broken during manufacture is prevented. Note that the compressed lines may be formed by adopting a configuration in which dot-shaped recess portions are intermittently arranged, or the like, without using such a two-step compression structure, depending on the strength and compression amount of the top sheet 14.

Meanwhile, in FIG. 16, regions indicated by the broken lines surrounding the recess portions 22 are semi-compressed regions 25. More specifically, when the top sheet 14 and the absorber 13 are firmly compressed to form the recess portions 22, the top sheet 14 and the absorber 13 around the recess portions 22 are pulled and compressed. Therefore, the absorber 13 is compressed in the semi-compressed regions 25 compared with the uncompressed regions such as the intersection regions 21D and the main absorptive regions 24.

As shown in FIG. 17, the absorber 13 has a high density portion 13A with the highest density of the absorber 13, in the bottom portion of the second recess portion 222. The uncompressed regions such as the main absorptive regions 24 are low density portions 13D with the lowest density of the absorber 13. The bottom portion of the first recess portion 221 is a medium density portion 13B. The semi-compressed region 25 is a density changing portion 13C in which the density is gradually reduced toward the uncompressed region.

Here, the absorber 13 is mainly made of SAPs and pulp as described above, and pulp fiber density is mainly responsible for the density thereof. Therefore, the high density portion 13A is in a state where the pulp is compressed with fewer gaps between pulp fibers. On the other hand, the low density portion 13D is in a state where there are more gaps between pulp fibers than the high density portion 13A.

In addition, the recess portions 22 are formed by compressing and joining together the top sheet 14 made of synthetic fiber and the absorber 13 mainly made of pulp. Thus, it is considered that the bottom 22a of each of the recess portions 22 is turned into a film, which hardly absorbs a liquid. Therefore, the body fluid discharged onto the recess portion 22 is absorbed by the density changing portion 13C that is the sidewall of the recess portion 22, and is then sent to the main absorptive region 24.

In this embodiment, no recess portions 22 are provided in the intersection regions 21D in the oblique lattice emboss pattern described above. As described above, the recess portions 22 are formed by rotating and pressing the embossing roll having a mold formed on its surface, the mold corresponding to the recess portions 22. As shown in FIG. 15, when the embossing roll travels in the arrow W direction while rotating, the recess portions 22 arranged in the direction perpendicular to the traveling direction W of the embossing roll are all pressed at the same time. In this embodiment, the size of the embossing roll is determined so that the circumference of the embossing roll corresponds to the longitudinal length of the absorber 13. However, the present invention is not limited thereto.

Note that, in this embodiment, the traveling direction W of the embossing roll is parallel to the direction of the imaginary line P described above, when the diaper 10 is completed. As described above, the top sheet 14 and the absorber 13 are both compressed by the embossing roll and then cut into the size of the diaper 10 by laminating another sheet required or the like. Since the cutting is performed over the length across the front body, the crotch, and the back body of the diaper 10, the oblique lattice emboss pattern tilted with respect to the imaginary line P can be formed by also setting the traveling direction W of the emboss roll parallel to the imaginary line P.

Therefore, with respect to the traveling direction W, again, the rightward recess portions 22c are tilted to the right and the leftward recess portions 22d are tilted to the left. Thus, the distance between the rightward recess portion 22c and the leftward recess portion 22d, which are adjacent to each other, is reduced toward the intersection region 21D as the embossing roll travels in the traveling direction W.

Here, during pressing of the embossing roll to form the recess portions 22, the top sheet 14 is pulled so as to be drawn into the recess portions 22. Since the adjacent rightward and leftward recess portions 22c and 22d are simultaneously formed by the pressing, the top sheet 14 positioned therebetween are pulled from the left and right sides. More specifically, in FIG. 15, drawing forces indicated by the arrows B are simultaneously applied on the left and right sides to pull the top sheet 14. The shorter the distance between the adjacent rightward and leftward recess portions 22c and 22d, the smaller the margin portion of the top sheet 14 against the pulling by the drawing forces A. Thus, the top sheet 14 gradually becomes a very taut state.

For example, as for formation of lattices of the same size, assuming that a shape is formed in which the rightward recess portion 22c and the leftward recess portion 22d are connected at a corner of the lattice without the interval L2, the rightward recess portion 22c and the leftward recess portion 22d are disposed close to each other in the vicinity of the corner of the lattice. Also, the semi-compressed regions 25 thereof overlap with each other. Then, in the vicinity of the corner, the absorber 13 itself has a high density and is hard. In addition, the drawing force of the adjacent left and right recess portions 22d and 22c sets the top sheet 14 in a very taut state. Therefore, the top sheet 14 in the main absorptive region 24 near the corner comes into contact in the very taut state with the absorber 13. The SAPs in the absorber 13 come into contact with the top sheet 14, making the surface of the top sheet 14 feel rough. This prevents irritation of the skin of the wearer, which is not preferable.

Therefore, in this embodiment, the very taut state of the top sheet 14 is prevented by not compressing the lattice intersection portion where the distance between the adjacent recess portions 22 is less than a predetermined distance T, i.e., the corner portion of the lattice square. More specifically, the compression of the absorber 13 by the recess portions 22 and the junction between the absorber 13 and the top sheet 14 are performed to the extent that the top sheet 14 in the main absorptive regions 24 can be maintained in a comfortably tense state. Thus, the rough texture due to the SAPs and hardening of the corner portions in the lattice pattern are suppressed.

More specifically, as shown in FIGS. 15 and 16, assuming that the shortest distance between the rightward recess portion 22c and the leftward recess portion 22d is T, the recess portions 22 are formed only in a portion where the distance therebetween is T or more. In other words, the recess portions 22 are formed only in a portion where the distance between the two sides that form the corner of the square is T or more, and no recess portions 22 are formed in a portion where the distance is less than T. Therefore, the intersection regions 21D (corner portions of the square and the vicinities thereof) are not compressed, and thus become uncompressed regions where the absorber 13 has the same thickness as the main absorptive region 24. Moreover, the top sheet 14 is fixed in a fluffy state. In FIGS. 15 and 16, the distance T is illustrated in the direction perpendicular to the traveling direction W. However, since a problem also arises from hardening of the corner portions in the direction parallel to the traveling direction W, the recess portions 22 are preferably formed spaced apart by a certain distance or more. In this embodiment, the rightward and leftward recess portions 22c and 22d have the same length, and the formation interval L2 therebetween are set equal. Thus, the recess portions 22 are similarly formed spaced apart by the distance T or more in the direction parallel to the traveling direction W.

In other words, to determine the emboss pattern, the arrangement interval between the recess portions 22 is determined such that the distance between the adjacent recess portions is T or more in the direction parallel to the diagonal line of the rectangular shape (square) defined by the first and second compressed lines 23a and 23b.

Note that, as described above, the drawing force A acts strongly in the direction perpendicular to the traveling direction W of the embossing roll. Thus, it can also be said that the diagonal line parallel to the traveling direction W of the embossing roll is not affected much even if the distance between the adjacent recess portions is slightly shorter than T.

In this embodiment, T is about 7 mm, for example, when the thickness of the absorber 13 is 8 mm and the depth Q12 of the first recess portion 221 is 6 mm. This also varies with the relationship between the SAP amount and the pulp fiber amount in the absorber 13 as well as the thickness of the top sheet 14. It is important in the present invention that no emboss pattern is formed in the portion where the interval between the adjacent recess portions is small in the case of using an emboss pattern with oblique components, such as an oblique lattice pattern.

As described above, in this embodiment, the shortest distance between the adjacent recess portions 22 is prevented from falling below T. More specifically, the skin contact surface is prevented from feeling rough by adopting an emboss pattern in which the recess portions 22 are arranged spaced apart by the distance T or more, i.e., an emboss pattern in which the vicinities of intersection points in the lattice pattern are not embossed. Moreover, the lattice corners are set in a fluffy state as in the case of the main absorptive region 24. Thus, even with the oblique lattice emboss pattern, the lattice corners do not feel rough and sharp, thus making it possible to give a fluffy texture to the wearer. Therefore, by providing no recess portions 22 in the intersection regions 21D between the first and second compressed lines 23a and 23b, the lattice corners do not become rough and sharp, and the absorber 13 in the intersection regions 21D have the same thickness and height as those in the main absorptive region 24. Thus, even when the oblique lattice emboss pattern is formed, the skin contact surface of the diaper 10 gives a fluffy texture to the wearer as a whole, and the lattice corner portions do not irritate the skin.

Note that, in this embodiment, the description is given of the case where the embossing is performed with the embossing roll. However, the present invention is not limited to this method. Alternatively, an embossing plate in the form of a plate corresponding to the size of the diaper 10 may be used to perform embossing by the size of the diaper 10.

Next, detailed description is given of an example of an emboss pattern by embossing.

As shown in FIG. 15, in the emboss pattern of this example, the rightward recess portions 22c form the first compressed line 23a with the intervals L2 at the front and back in the longitudinal direction thereof, and the leftward recess portions 22d form the second compressed line 23b with the intervals L2 at the front and back in the longitudinal direction thereof. The first compressed lines 23a are arranged in parallel at the interval S1, while the second compressed lines 23b are arranged in parallel at the interval S2. Such first and second compressed lines 23a and 23b form the emboss pattern that is the oblique lattice pattern when seen from a distance, as shown in FIG. 15.

In this example, each side of the lattice has an equal length S1 or S2, which is 27.0 mm. The interval of the lattice is preferably 13.0 mm to 54.0 mm. Also, the length U in the longitudinal direction of the recess portion 22 is preferably about 55% to 85% of the length S1 or S2 of each side of the lattice. In this example, the length U is 19.0 mm. Meanwhile, the width V of the recess portion 22 is 3.0 mm. The width V is preferably 2.0 mm to 4.0 mm. Moreover, the interval L2 between the recess portions 22 is 8.8 mm. The interval L2 is preferably 4.0 mm to 12.0 mm. Such relationships lead to a state where no recess portions are formed in spots where the distance T between the adjacent recess portions 22 is less than 6.0 mm.

Moreover, in this embodiment, the thickness of the absorber 13 and the top sheet 14 before embossing is 8.0 mm. As shown in FIG. 17, the depth Q11 of the deepest spot in the recess portion 22 is 7.8 mm, and the depth Q12 of the second deepest spot therein is 7.5 mm. Also, the thickness Q14 of the uncompressed region is 8.0 mm.

Note that the thickness of the absorber 13 and the top sheet 14 before embossing is preferably 5.0 mm to 20.0 mm. The maximum depth Q11 of the portion compressed by the recess portions 22 is preferably 3.0 mm to 8.0 mm. A difference Q13 from the second deepest spot is preferably 0.1 mm to 0.5 mm. Also, the thickness Q14 of the uncompressed region is preferably 5.0 mm to 20.0 mm.

By forming the recess portions 22 with such depths and intervals, the grooves can be maintained even when the body weight is applied thereto, and a soft texture can be maintained on the skin contact surface in the crotch portion of the diaper 10. Therefore, the oblique lattice emboss pattern can suppress deformation of the diaper 10, such as twisting, against various movements of the legs, and can minimize irritation of the skin by achieving a soft texture.

Note that, in this embodiment, the recess portion 22 has its external shape determined by the first recess portion 221 having the approximately oval elongated shape. However, the present invention is not limited thereto. Alternatively, compressed lines in an oblique lattice pattern may be formed by intermittently arranging recess portions, each having a dot shape, a parallelogram shape or the like. More specifically, the same effects as those of this embodiment can be achieved by increasing the arrangement interval between the recess portions in the corner portion (intersection portion) in the oblique lattice square compared with the side portion of the square. In this case, the recess portions are arranged so that the distance between the adjacent recess portions in the direction parallel to the diagonal line of the lattice square is T or more.

Moreover, although one recess portion 22 forms one side of the square in this embodiment, the longitudinal length thereof may be reduced and one side of the square may be formed by two or more recess portions 22. Also, as for the arrangement of the second recess portions 222 within the first recess portion 221, the second recess portions 222 are alternately allocated on the outside in the width direction. However, the present invention is not limited thereto, but any configuration may be adopted, such as one in which the second recess portions 222 are lined up in the center. Moreover, the shape of the second recess portion 222 is not limited to that in this embodiment, but various shapes such as a square shape, an oval shape, and a triangular shape may be adopted.

The disposable diaper 10 according to this embodiment is applicable to both adults and children. Moreover, in this embodiment, the description is given of, as an example, the unfolding-type disposable diaper 10 (so-called sheet-type disposable diaper). However, it is needless to say that the disposable diaper 10 according to this embodiment is also applicable to a pants-type diaper. Furthermore, the absorbent article according to the present invention is not specified as a diaper only, but is applicable to other general absorbent articles such as an absorbent pad. For example, the present invention is also applicable to an absorbent pad or the like based on the structure of the portion including the absorber 13 and the top sheet 14 as shown in FIG. 14. The same advantageous effects as those of the diaper are achieved.

Next, description is given of a relationship between the method for forming recess portions and the thickness of the intersection region. The recess portions 22 are formed by compressing the top surface sheet 14 and the absorber 13 with molds from the top surface sheet 14 side of the disposable diaper 10, for example.

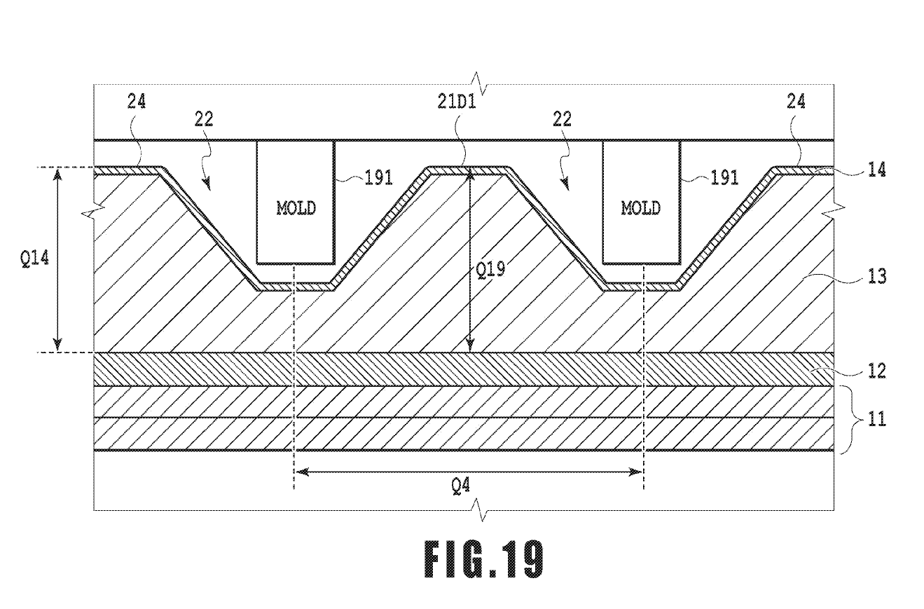

FIG. 19 is a view showing a method for forming recess portions when there is a large interval between adjacent molds. When an interval between adjacent molds 191, i.e., an interval Q4 between the recess portions 22 across an intersection region 21D1 is large, hardly any force is applied to the surface of the intersection region 21D1 from both sides thereof, which is positioned between the recess portions 22 compressed by the molds 191. Thus, the thickness Q19 of the intersection region 21D1 does not change. Therefore, the thickness Q19 of the intersection region 21D1 is equal to the thickness Q14 of the main absorptive region 24.

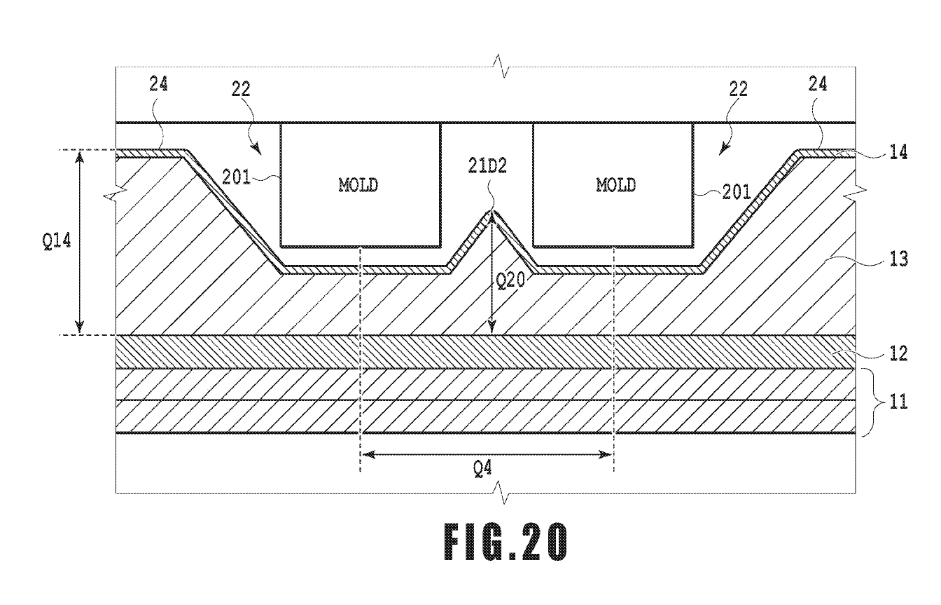

FIG. 20 is a view showing a method for forming recess portions when there is a small interval between adjacent molds. When an interval between adjacent molds 201, i.e., an interval Q4 between the recess portions 22 across an intersection region 21D2 is small, the top surface sheet 14 and the absorber 13 in the intersection region 21D2 are pushed into the intersection region 21D2 positioned between the recess portions 22 compressed by the molds 201 by compressing the top surface sheet 14 and the absorber 13 on both sides thereof. Thus, the recess portions are formed, which are not directly compressed. Therefore, a thickness Q20 of the intersection region 21D2 is smaller than the thickness Q14 of the main absorptive region 24.

Note that FIG. 2 shows a standing state of the lower part of the baby's body wearing the diaper 10. However, the present invention is not aimed at only the case of standing and walking, but achieves the advantageous effects for general back and forth movements of the legs, such as rolling over and crawling of a baby.

The structure of the diaper to be the target of the present invention is not limited to the unfolding type as described above. The present invention is applicable to any configuration as long as the diaper includes the configuration of the absorbent article according to claims. For example, the present invention is also applicable to well-known pants-type disposable diaper, urine leakage prevention pad, and the like.

In addition, the present invention is not limited to a diaper for baby, but is applicable to various absorbent articles such as a diaper for adult and the urine leakage prevention pad.

An absorbent article according to the present invention includes a liquid permeable top surface sheet, a liquid impermeable back surface sheet, and an absorber disposed between the top surface sheet and the back surface sheet. The absorbent article may include a flow channel formation region including a plurality of flow channels extending on the top surface sheet. The flow channel may include a plurality of recess portions formed by compressing the top surface sheet and the absorber.

In the absorbent article according to the present invention, the recess portions may be intermittently arranged at the bottom of the flow channel.

In the absorbent article according to the present invention, no recess portions may be positioned in intersection regions of the plurality of flow channels.

In the absorbent article according to the present invention, a pressed density of the top surface sheet and the absorber in the intersection regions of the plurality of flow channels may be lower than that of the top surface sheet and the absorber in the bottom of the recess portion.

In the absorbent article according to the present invention, the flow channels may intermittently or continuously extend from one end to the other end of the flow channel formation region.

In the absorbent article according to the present invention, the flow channel formation region may not reach the end of the absorber.

In the absorbent article according to the present invention, an interval between the adjacent recess portions may be 10 mm to 70 mm.

The absorbent article according to the present invention may be a disposable diaper in which the absorber has a size that extends across the front body, the crotch, and the back body.

An absorbent article according to the present invention includes a liquid permeable top surface sheet, a liquid impermeable back surface sheet, and an absorber disposed between the top surface sheet and the back surface sheet, the absorber containing pulp fibers and SAPs. The top surface sheet side of the absorbent article may include a flat portion and a plurality of recess portions formed by integrally joining the top surface sheet and the absorber. The plurality of recess portions may form a plurality of flow channels for passing a liquid by partitioning the flat portion into a plurality of flat sections. The partitioned flat sections may each have an area of 0.5 cm² or more.

In the absorbent article according to the present invention, the liquid permeability of the flow channels may be lower than that of the flat portion.

In the absorbent article according to the present invention, the SAP content of the absorber per unit area may be 100 g/cm² or more.

In the absorbent article according to the present invention, the width of the flow channel may be 1.0 mm to 6.0 mm.

In the absorbent article according to the present invention, the area of the flat section may be 0.5 cm² to 50.0 cm².

In the absorbent article according to the present invention, the plurality of flow channels may extend intermittently or continuously in a lattice pattern.

In the absorbent article according to the present invention, the flow channels maybe formed by compressing the top surface sheet and the absorber together.

The absorbent article according to the present invention may be a disposable diaper of a size that extends across the front body, the crotch, and the back body.

An absorbent article according to the present invention includes a liquid permeable top surface sheet, a liquid impermeable back surface sheet, an absorber disposed between the top surface sheet and the back surface sheet, and a compressed section formation region including a plurality of compressed lines in which a plurality of recess portions are arranged, the recess portions being formed by compressing the top surface sheet and the absorber. The compressed lines may include: a first compressed line extending while being tilted to one side with respect to an imaginary line extending from the abdomen side of the wearer when he/she wears the absorbent article to the dorsal side through the crotch; and a second compressed line extending while being tilted to the other side. In the compressed section formation region, a plurality of the first and second compressed lines maybe arranged in a lattice pattern, and no recess portions may be provided in intersection portions between the first and second compressed lines.

In the absorbent article according to the present invention, the recess portions maybe formed by integrally joining the top surface sheet and the absorber.

In the absorbent article according to the present invention, uncompressed regions in the absorber may all have the same thickness, and the intersection portion may be the uncompressed region.

In the absorbent article according to the present invention, a distance between the adjacent recess portions may be 6 mm or more in a direction parallel to a diagonal line of a rectangular shape defined by the first and second compressed lines.

In the absorbent article according to the present invention, an interval between the plurality of first compressed lines and an interval between the plurality of second compressed lines may be 13 mm to 54 mm.

In the absorbent article according to the present invention, the recess portion may include a first recess portion and a second recess portion that is further depressed within the first recess portion.

The absorbent article according to the present invention may be a disposable diaper in which the absorber has a size that extends across the front body, the crotch, and the back body.

### REFERENCE SIGNS LIST

10 disposable diaper
10A fastening tape
10B front patch sheet
10F front body region
10R back body region
10C crotch region
10W waist opening
10L leg opening
10D elastic sheet
11 cover sheet
11A cutout
11B inner cover sheet
11C outer cover sheet
12 back sheet (back surface sheet)
13 absorber
13S hydrophilic sheet
13T liquid diffusion sheet
14 top sheet (top surface sheet)
15, 19 rubber thread
18 side sheet
21 flow channel
21a first flow channel
21b second flow channel
21D, 21D1, 21D2 intersection region (intersection point of flow channels)
22 recess portion
22a bottom of recess portion
22b portion other than recess portion in flow channel (recess space)
22c rightward recess portion (recess portion tilted in first direction)
22d leftward recess portion (recess portion tilted in second direction)
221 first recess portion
222 second recess portion (portion forming deepest bottom in recess portion)
23 compressed line
23a first compressed line
23b second compressed line
24, 24A, 24B main absorptive region (flat section)
25 semi-compressed region
191, 201 mold
Q1 width of flow channel
Q2 diameter of recess portion
Q3 arrangement interval between recess portions 22
Q4 distance between centers of adjacent recess portions in intersection region
Q5 depth of recess portion 22
Q11 maximum depth of recess portion to bottom of second recess portion
Q12 depth to bottom of first recess portion
Q13 difference in depth between first recess portion and second recess portion
Q14 thickness of uncompressed region (main absorptive region)
Q19, Q20 interval between adjacent recess portions
L1 diagonal line in one square
L2 longitudinal interval between adjacent recess portions in compressed line
S1 formation interval between first compressed lines
S2 formation interval between second compressed lines
T shortest distance between adjacent recess portions
U long-side length of recess portion
V short-side length (width) of recess portion

1. An absorbent article comprising:
a liquid permeable top surface sheet;
a liquid impermeable back surface sheet; and
an absorber disposed between the top surface sheet and the back surface sheet, wherein
a plurality of compressed lines extend on a top surface sheet side of the absorbent article, and in each of the plurality of compressed lines, a plurality of recess portions formed by compressing the top surface sheet and the absorber are arranged,
the plurality of compressed lines form an intersection region where the compressed lines intersect with each other, and
the compressed recess portions are not formed in the intersection region.

2. The absorbent article according to claim 1, wherein
a pressed density of the top surface sheet and the absorber in the intersection region is lower than a pressed density of the top surface sheet and the absorber in the recess portions.

3. The absorbent article according to claim 1, wherein
the intersection region has the same thickness as a thickness of a main absorptive region surrounded by the plurality of compressed lines.

4. The absorbent article according to claim 1, wherein
the intersection region has a thickness smaller than a thickness of a main absorptive region surrounded by the plurality of compressed lines.

5. The absorbent article according to claim 1, wherein
a main absorptive region surrounded by the plurality of compressed lines has an area of 0.5 cm² or more.

6. The absorbent article according to claim 1, wherein
the plurality of compressed lines extend in an intermittent or continuous lattice pattern.

7. The absorbent article according to claim 1, wherein
the recess portions each include a first recess portion and a second recess portion that is further depressed in the first recess portion.
